(19) United States
(12) Patent Application Publication
(10) Pub. No.: US 2006/0142712 A1
(43) Pub. Date: Jun. 29, 2006

(54) Absorbent articles that provide warmth

(75) Inventor: Roger Bradshaw Quincy III, Cumming, GA (US)

Correspondence Address: DORITY & MANNING, P.A., POST OFFICE BOX 1449, GREENVILLE, SC, 29602-1449, US

(73) Assignee: Kimberly-Clark Worldwide, Inc.

(21) Appl. No.: 11/021,546

(22) Filed: Dec. 23, 2004

(51) Int. Cl. A61F 13/15

(52) U.S. Cl. 604/364

(57) ABSTRACT

An absorbent article that contains a warmth-providing substrate is provided that is capable of generating heat upon activation. Specifically, the substrate is coated with an exothermic composition that may be formed from a variety of different components, including oxidizable metals, carbon components, binders, electrolytic salts, and so forth. The oxidizable metal is capable of undergoing an exothermic, electrochemical reaction in the presence of oxygen and water to generate heat. In some cases, the exothermic composition is anhydrous, i.e., generally free of water, to reduce the likelihood of premature activation prior to use.

### BACKGROUND OF THE INVENTION

Absorbent articles, such as diapers, child training pants, adult incontinence garments, swim wear, and so forth, often include a liquid-permeable top layer for direct contact with the wearer, an absorbent core, and a substantially liquid-impermeable outer cover. The absorbent core is positioned between the top layer and the outer cover. When the absorbent article is exposed to a liquid insult, liquid passes through the top layer and into the absorbent core. The outer cover prevents the liquid in the absorbent core from leaving the garment. Many of today's absorbent garments utilize breathable outer cover materials. Breathable outer cover materials are substantially impermeable to liquids, but are permeable to water vapor. Such materials permit the escape of water vapor from the absorbent garment, thereby increasing comfort and reducing skin rashes and other irritations that may result when water vapor is trapped inside the garment. However, one common shortcoming of such breathable absorbent articles is that a cold, damp, and clammy feel may result on the outside of the garment, i.e., on the outside of the outer cover. Specifically, liquid water in the absorbent may evaporate and pass through the outer cover. The evaporation of water lowers the temperature of the absorbent and adjacent outer cover, thereby resulting in the cold, damp, and clammy feeling.

As such, a need exists for an absorbent article that remains breathable, but yet also avoids the perceived cold, damp, and clammy feeling associated with evaporative cooling.

### SUMMARY OF THE INVENTION

In accordance with one embodiment of the present invention, an absorbent article is disclosed that comprises a substantially liquid-impermeable layer, a liquid-permeable layer, and an absorbent positioned between the substantially liquid-impermeable layer and the liquid-permeable layer. The absorbent article also comprises an exothermic coating that is formed from an oxidizable metal powder and is capable of activation in the presence of oxygen and moisture to generate heat. Other ingredients may of course be utilized in the exothermic coating, such as a carbon component, a binder, an electrolytic salt, water-retaining particles, a pH adjuster, a surfactant, etc. Regardless, the composition is generally free of water prior to activation.

In accordance with another embodiment of the present invention, a personal care absorbent article is disclosed that comprises a liquid-permeable liner, a breathable outer cover, an absorbent positioned between the liner and the outer cover, and optionally, a ventilation layer positioned between the breathable outer cover and the absorbent. The breathable outer cover, ventilation layer, or both, comprise an exothermic coating that is formed from an oxidizable metal powder and is capable of activation in the presence of oxygen and moisture to generate heat. Prior to activation, the exothermic coating is generally free of water.

In accordance with still another embodiment of the present invention, a diaper is disclosed that comprises a liquid-permeable bodyside liner, a breathable outer cover, an absorbent positioned between the liner and the outer cover, a surge layer positioned between the liner and the absorbent, and optionally, a ventilation layer positioned between the outer cover and the absorbent. The breathable outer cover, ventilation layer, or both, comprise an exothermic coating that is formed from an oxidizable metal powder, carbon component, binder, and metal halide. The exothermic coating is capable of activation in the presence of oxygen and moisture to generate heat. Prior to activation, the exothermic coating is generally free of water.

Other features and aspects of the present invention are described in more detail below.

### BRIEF DESCRIPTION OF THE DRAWINGS

A full and enabling disclosure of the present invention, including the best mode thereof, directed to one of ordinary skill in the art, is set forth more particularly in the remainder of the specification, which makes reference to the appended figure in which:

FIG. 1 illustrates a perspective view of an absorbent article that may be formed according to one embodiment of the present invention; and

FIG. 2 is a thermal response curve showing temperature versus time for the samples of Example 2.

### DETAILED DESCRIPTION OF REPRESENTATIVE EMBODIMENTS

### Definitions

As used herein, an “absorbent article” refers to any article capable of absorbing water or other fluids. Examples of some absorbent articles include, but are not limited to, personal care absorbent articles, such as diapers, training pants, absorbent underpants, adult incontinence products, feminine hygiene products (e.g., sanitary napkins), swim wear, baby wipes, and so forth; medical absorbent articles, such as garments, fenestration materials, underpads, bandages, absorbent drapes, and medical wipes; food service wipers; clothing articles; and so forth. Materials and processes suitable for forming such absorbent articles are well known to those skilled in the art.

As used herein the term “nonwoven fabric or web” means a web having a structure of individual fibers or threads which are interlaid, but not in an identifiable manner as in a knitted fabric. Nonwoven fabrics or webs have been formed from many processes such as for example, meltblowing processes, spunbonding processes, bonded carded web processes, etc.

As used herein, the term “meltblowing” refers to a process in which fibers are formed by extruding a molten thermoplastic material through a plurality of fine, usually circular, die capillaries as molten fibers into converging high velocity gas (e.g. air) streams that attenuate the fibers of molten thermoplastic material to reduce their diameter, which may be to microfiber diameter. Thereafter, the meltblown fibers are carried by the high velocity gas stream and are deposited on a collecting surface to form a web of randomly disbursed meltblown fibers. Such a process is disclosed, for example, in U.S. Pat. No. 3,849,241 to Butin, et al., which is incorporated herein in its entirety by reference thereto for all purposes. Generally speaking, meltblown fibers may be microfibers that may be continuous or discontinuous, are generally smaller than 10 microns in diameter, and are generally tacky when deposited onto a collecting surface.

As used herein, the term “spunbonding” refers to a process in which small diameter substantially continuous fibers are formed by extruding a molten thermoplastic material from a plurality of fine, usually circular, capillaries of a spinnerette with the diameter of the extruded fibers then being rapidly reduced as by, for example, eductive drawing and/or other well-known spunbonding mechanisms. The production of spun-bonded nonwoven webs is described and illustrated, for example, in U.S. Pat. No. 4,340,563 to Appel, et al., U.S. Pat. No. 3,692,618 to Dorschner, et al., U.S. Pat. No. 3,802,817 to Matsuki, et al., U.S. Pat. No. 3,338,992 to Kinney, U.S. Pat. No. 3,341,394 to Kinney, U.S. Pat. No. 3,502,763 to Hartman, U.S. Pat. No. 3,502,538 to Levy, U.S. Pat. No. 3,542,615 to Dobo, et al., and U.S. Pat. No. 5,382,400 to Pike, et al., which are incorporated herein in their entirety by reference thereto for all purposes. Spunbond fibers are generally not tacky when they are deposited onto a collecting surface. Spunbond fibers may sometimes have diameters less than about 40 microns, and are often between about 5 to about 20 microns.

As used herein, the term “coform” generally refers to composite materials comprising a mixture or stabilized matrix of thermoplastic fibers and a second non-thermoplastic material. As an example, coform materials may be made by a process in which at least one meltblown die head is arranged near a chute through which other materials are added to the web while it is forming. Such other materials may include, but are not limited to, fibrous organic materials such as woody or non-woody pulp such as cotton, rayon, recycled paper, pulp fluff and also superabsorbent particles, inorganic and/or organic absorbent materials, treated polymeric staple fibers and so forth. Some examples of such coform materials are disclosed in U.S. Pat. Nos. 4,100,324 to Anderson, et al.; U.S. Pat. No. 5,284,703 to Everhart, et al.; and U.S. Pat. No. 5,350,624 to Georger, et al.; which are incorporated herein in their entirety by reference thereto for all purposes.

As used herein, the “water vapor transmission rate” (WVTR) generally refers to the rate at which water vapor permeates through a material as measured in units of grams per meter squared per 24 hours (g/m²/24 hrs). The test used to determine the WVTR of a material may vary based on the nature of the material. For instance, in some embodiments, WVTR may be determined in general accordance with ASTM Standard E-96E-80. This test may be particularly well suited for materials thought to have a WVTR of up to about 3,000 g/m²124 hrs. Another technique for measuring WVTR involves the use of a PERMATRAN-W 100K water vapor permeation analysis system, which is commercially available from Modern Controls, Inc. of Minneapolis, Minn. Such a system may be particularly well suited for materials thought to have a WVTR of greater than about 3,000 gm²/24 hrs. However, as is well known in the art, other systems and techniques for measuring WVTR may also be utilized.

As used herein, the term “breathable” means pervious to water vapor and gases, but impermeable to liquid water. For instance, “breathable barriers” and “breathable films” allow water vapor to pass therethrough, but are substantially impervious to liquid water. The “breathability” of a material is measured in terms of water vapor transmission rate (WVTR), with higher values representing a more vapor-pervious material and lower values representing a less vapor-pervious material. Typically, after being coated with an exothermic coating, the “breathable” materials have a water vapor transmission rate (WVTR) of at least about 100 grams per square meter per 24 hours (g/m²/24 hours), in some embodiments from about 500 to about 20,000 g/m²124 hours, and in some embodiments, from about 1,000 to about 15,000 g/m²124 hours.

### DETAILED DESCRIPTION

Reference now will be made in detail to various embodiments of the invention, one or more examples of which are set forth below. Each example is provided by way of explanation, not limitation of the invention. In fact, it will be apparent to those skilled in the art that various modifications and variations may be made in the present invention without departing from the scope or spirit of the invention. For instance, features illustrated or described as part of one embodiment, may be used on another embodiment to yield a still further embodiment. Thus, it is intended that the present invention cover such modifications and variations.

In general, the present invention is directed to an absorbent article that contains a warmth-providing substrate, which is capable of generating heat upon activation. Specifically, the substrate contains an exothermic coating that may be formed from a variety of different components, including oxidizable metals, carbon components, binders, electrolytic salts, and so forth. The oxidizable metal is capable of undergoing an exothermic reaction in the presence of oxygen and moisture to generate heat. In some cases, the exothermic coating is anhydrous, i.e., generally free of water, to reduce the likelihood of premature activation prior to use.

The warmth-providing substrate of the present invention may form the entire absorbent article, or may form only a portion of the article. For example, the absorbent article generally includes a substantially liquid-impermeable layer (e.g., outer cover), a liquid-permeable layer (e.g., bodyside liner, surge layer, etc.), and an absorbent. During use, moisture is initially received by the liquid-permeable layer and transferred to the absorbent. However, the moisture retained by the absorbent may generate vapors that migrate through the substantially liquid-impermeable layer, particularly when it is pervious to vapors and gases, i.e., “breathable.” Thus, the vapors may condense on the surface of the substantially liquid-impermeable layer and create a cool and damp sensation to the wearer. The present inventor has discovered that such a cool and damp sensation may be mitigated by a warmth-providing substrate. For example, the warmth-providing substrate may form part or all of a substantially liquid-impermeable layer. When utilized in this manner, the substrate may not only provide warmth, but also function in its normal capacity for the absorbent article. For instance, outer covers are generally configured to allow the release of vapors from the absorbent core. When utilized in the outer cover, the warmth-providing substrate of the present invention may still function in this manner.

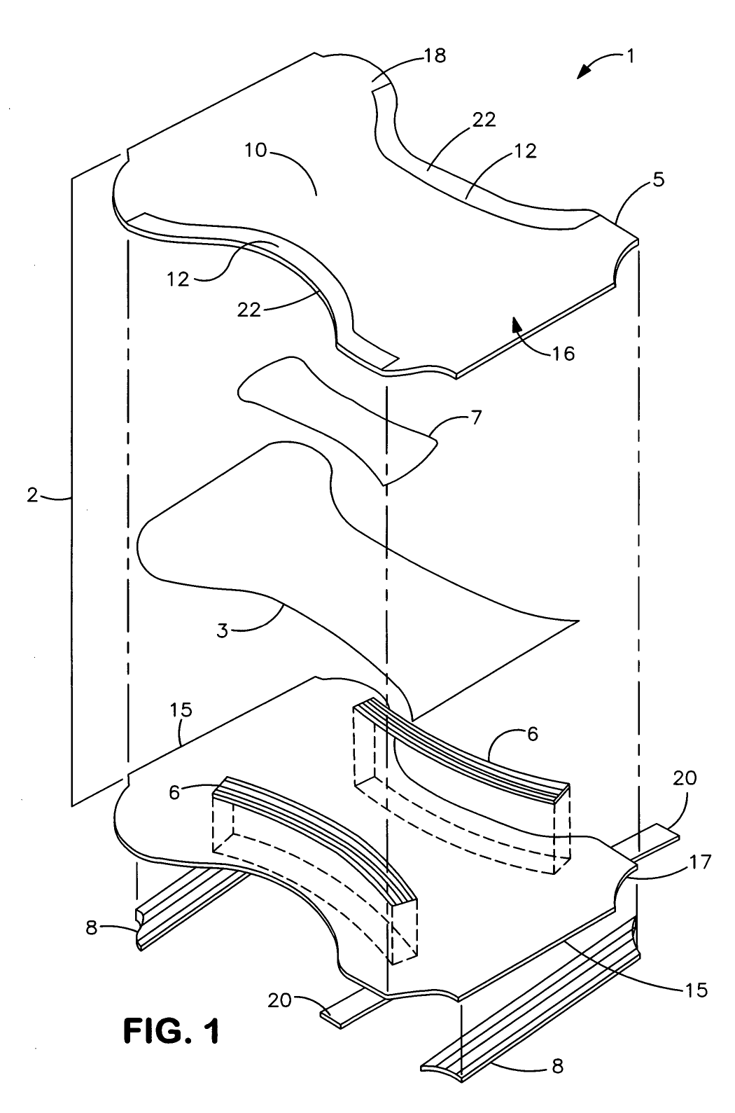

In this regard, various embodiments of an absorbent article that may be formed according to the present invention will now be described in more detail. For purposes of illustration only, an absorbent article is shown in FIG. 1 as a diaper 1. However, as discussed above, the invention may be embodied in other types of absorbent articles, such as sanitary napkins, diaper pants, feminine napkins, children's training pants, and so forth. In the illustrated embodiment, the diaper 1 is shown as having an hourglass shape in an unfastened configuration. However, other shapes may of course be utilized, such as a generally rectangular shape, T-shape, or I-shape. As shown, the diaper 1 includes a chassis 2 formed by various components, including an outer cover 17, bodyside liner 5, absorbent core 3, and surge layer 7. It should be understood, however, that other layers may also be used in the present invention. Likewise, one or more of the layers referred to in FIG. 1 may also be eliminated in certain embodiments of the present invention.

The outer cover 17 is typically formed from a material that is substantially impermeable to liquids. For example, the outer cover 17 may be formed from a thin plastic film or other flexible liquid-impermeable material. In one embodiment, the outer cover 17 is formed from a polyethylene film having a thickness of from about 0.01 millimeter to about 0.05 millimeter. If a more cloth-like feeling is desired, the outer cover 17 may be formed from a polyolefin film laminated to a nonwoven web. For example, a stretch-thinned polypropylene film having a thickness of about 0.015 millimeter may be thermally laminated to a spunbond web of polypropylene fibers. The polypropylene fibers may have a denier per filament of about 1.5 to 2.5, and the nonwoven web may have a basis weight of about 17 grams per square meter (0.5 ounce per square yard). The outer cover 17 may also include bicomponent fibers, such as polyethylene/polypropylene bicomponent fibers.

In addition, the outer cover 17 may also be formed from a material that is impermeable to liquids, but permeable to gases and water vapor (i.e., “breathable”). This permits vapors to escape from the absorbent core 3, but still prevents liquid exudates from passing through the outer cover 17. For example, the outer cover 17 may contain a breathable film, such as a microporous or monolithic film. The film may be formed from a polyolefin polymer, such as linear, low-density polyethylene (LLDPE) or polypropylene. Examples of predominately linear polyolefin polymers include, without limitation, polymers produced from the following monomers: ethylene, propylene, 1-butene, 4-methyl-pentene, 1-hexene, 1-octene and higher olefins as well as copolymers and terpolymers of the foregoing. In addition, copolymers of ethylene and other olefins including butene, 4-methyl-pentene, hexene, heptene, octene, decene, etc., are also examples of predominately linear polyolefin polymers.

If desired, the breathable film may also contain an elastomeric polymer, such as elastomeric polyesters, elastomeric polyurethanes, elastomeric polyamides, elastomeric polyolefins, elastomeric copolymers, and so forth. Examples of elastomeric copolymers include block copolymers having the general formula A-B-A′ or A-B, wherein A and A′ are each a thermoplastic polymer endblock that contains a styrenic moiety (e.g., poly(vinyl arene)) and wherein B is an elastomeric polymer midblock, such as a conjugated diene or a lower alkene polymer (e.g., polystyrene-poly(ethylene-butylene)-polystyrene block copolymers). Also suitable are polymers composed of an A-B-A-B tetrablock copolymer, such as discussed in U.S. Pat. No. 5,332,613 to Taylor, et al., which is incorporated herein in its entirety by reference thereto for all purposes. An example of such a tetrablock copolymer is a styrene-poly(ethylene-propylene)-styrene-poly(ethylene-propylene) (“S-EP-S-EP”) block copolymer. Commercially available A-B-A′ and A-B-A-B copolymers include several different formulations from Kraton Polymers of Houston, Tex. under the trade designation KRATON®. KRATON® block copolymers are available in several different formulations, a number of which are identified in U.S. Pat. Nos. 4,663,220, 4,323,534, 4,834,738, 5,093,422 and 5,304,599, which are hereby incorporated in their entirety by reference thereto for all purposes. Other commercially available block copolymers include the S-EP-S or styrene-poly(ethylene-propylene)-styrene elastomeric copolymer available from Kuraray Company, Ltd. of Okayama, Japan, under the trade name SEPTON®.

Examples of elastomeric polyolefins include ultra-low density elastomeric polypropylenes and polyethylenes, such as those produced by “single-site” or “metallocene” catalysis methods. Such elastomeric olefin polymers are commercially available from ExxonMobil Chemical Co. of Houston, Tex. under the trade designations ACHIEVE® (propylene-based), EXACT® (ethylene-based), and EXCEED® (ethylene-based). Elastomeric olefin polymers are also commercially available from DuPont Dow Elastomers, LLC (a joint venture between DuPont and the Dow Chemical Co.) under the trade designation ENGAGE® (ethylene-based) and AFFINITY® (ethylene-based). Examples of such polymers are also described in U.S. Pat. Nos. 5,278,272 and 5,272,236 to Lai, et al., which are incorporated herein in their entirety by reference thereto for all purposes. Also useful are certain elastomeric polypropylenes, such as described in U.S. Pat. No. 5,539,056 to Yang, et al. and U.S. Pat. No. 5,596,052 to Resconi, et al., which are incorporated herein in their entirety by reference thereto for all purposes.

If desired, blends of two or more polymers may also be utilized to form the breathable film. For example, the film may be formed from a blend of a high performance elastomer and a lower performance elastomer. A high performance elastomer is generally an elastomer having a low level of hysteresis, such as less than about 75%, and in some embodiments, less than about 60%. Likewise, a low performance elastomer is generally an elastomer having a high level of hysteresis, such as greater than about 75%. The hysteresis value may be determined by first elongating a sample to an ultimate elongation of 50% and then allowing the sample to retract to an amount where the amount of resistance is zero. Particularly suitable high performance elastomers may include styrenic-based block copolymers, such as described above and commercially available from Kraton Polymers of Houston, Tex. under the trade designation KRATON®). Likewise, particularly suitable low performance elastomers include elastomeric polyolefins, such as metallocene-catalyzed polyolefins (e.g., single site metallocene-catalyzed linear low density polyethylene) commercially available from DuPont Dow Elastomers, LLC under the trade designation AFFINITY®. In some embodiments, the high performance elastomer may constitute from about 25 wt. % to about 90 wt. % of the polymer component of the film, and the low performance elastomer may likewise constitute from about 10 wt. % to about 75 wt. % of the polymer component of the film. Further examples of such a high performance/low performance elastomer blend are described in U.S. Pat. No. 6,794,024 to Walton, et al., which is incorporated herein in its entirety by reference thereto for all purposes.

As stated, the breathable film may be microporous. The micropores form what is often referred to as tortuous pathways through the film. Liquid contacting one side of the film does not have a direct passage through the film. Instead, a network of microporous channels in the film prevents liquids from passing, but allows gases and water vapor to pass. Microporous films may be formed from a polymer and a filler (e.g., calcium carbonate). Fillers are particulates or other forms of material that may be added to the film polymer extrusion blend and that will not chemically interfere with the extruded film, but which may be uniformly dispersed throughout the film. Generally, on a dry weight basis, based on the total weight of the film, the film includes from about 30% to about 90% by weight of a polymer. In some embodiments, the film includes from about 30% to about 90% by weight of a filler. Examples of such films are described in U.S. Pat. No. 5,843,057 to McCormack; U.S. Pat. No. 5,855,999 to McCormack; U.S. Pat. No. 5,932,497 to Morman, et al.; U.S. Pat. No. 5,997,981 to McCormack et al.; U.S. Pat. No. 6,002,064 to Kobylivker. et al.; U.S. Pat. No. 6,015,764 to McCormack, et al.; U.S. Pat. No. 6,037,281 to Mathis, et al.; U.S. Pat. No. 6,111,163 to McCormack, et al.; and U.S. Pat. No. 6,461,457 to Taylor. et al., which are incorporated herein in their entirety by reference thereto for all purposes.

The films are generally made breathable by stretching the filled films to create the microporous passageways as the polymer breaks away from the filler (e.g., calcium carbonate) during stretching. For example, the breathable material contains a stretch-thinned film that includes at least two basic components, i.e., a polyolefin polymer and filler. These components are mixed together, heated, and then extruded into a film layer using any one of a variety of film-producing processes known to those of ordinary skill in the film processing art. Such film-making processes include, for example, cast embossed, chill and flat cast, and blown film processes.

Another type of breathable film is a monolithic film that is a nonporous, continuous film, which because of its molecular structure, is capable of forming a liquid-impermeable, vapor-permeable barrier. Among the various polymeric films that fall into this type include films made from a sufficient amount of poly(vinyl alcohol), polyvinyl acetate, ethylene vinyl alcohol, polyurethane, ethylene methyl acrylate, and ethylene methyl acrylic acid to make them breathable. Without intending to be held to a particular mechanism of operation, it is believed that films made from such polymers solubilize water molecules and allow transportation of those molecules from one surface of the film to the other. Accordingly, these films may be sufficiently continuous, i.e., nonporous, to make them substantially liquid-impermeable, but still allow for vapor permeability.

Breathable films, such as described above, may constitute the entire breathable material, or may be part of a multilayer film. Multilayer films may be prepared by cast or blown film coextrusion of the layers, by extrusion coating, or by any conventional layering process. Further, other breathable materials that may be suitable for use in the present invention are described in U.S. Pat. No. 4,341,216 to Obenour; U.S. Pat. No. 4,758,239 to Yeo, et al.; U.S. Pat. No. 5,628,737 to Dobrin. et al.; U.S. Pat. No. 5,836,932 to Buell; U.S. Pat. No. 6,114,024 to Forte; U.S. Pat. No. 6,153,209 to Vega, et al.; U.S. Pat. No. 6,198,018 to Curro; U.S. Pat. No. 6,203,810 to Alemany, et al.; and U.S. Pat. No. 6,245,401 to Ying, et al., which are incorporated herein in their entirety by reference thereto for all purposes.

If desired, the breathable film may also be bonded to a nonwoven web, knitted fabric, and/or woven fabric using well-known techniques. For instance, suitable techniques for bonding a film to a nonwoven web are described in U.S. Pat. No. 5,843,057 to McCormack; U.S. Pat. No. 5,855,999 to McCormack; U.S. Pat. No. 6,002,064 to Kobylivker, et al.; U.S. Pat. No. 6,037,281 to Mathis, et al.; and WO 99/12734, which are incorporated herein in their entirety by reference thereto for all purposes. For example, a breathable film/nonwoven laminate material may be formed from a nonwoven layer and a breathable film layer. The layers may be arranged so that the breathable film layer is attached to the nonwoven layer. In one particular embodiment, the breathable material is formed from a nonwoven fabric (e.g., polypropylene spunbonded web) laminated to a breathable film.

As stated, the diaper 1 also includes a bodyside liner 5. The bodyside liner 5 is generally employed to help isolate the wearer's skin from liquids held in the absorbent core 3. For example, the liner 5 presents a bodyfacing surface that is typically compliant, soft feeling, and non-irritating to the wearer's skin. Typically, the liner 5 is also less hydrophilic than the absorbent core 3 so that its surface remains relatively dry to the wearer. The liner 5 may be liquid-permeable to permit liquid to readily penetrate through its thickness.

The bodyside liner 5 may be formed from a wide variety of materials, such as porous foams, reticulated foams, apertured plastic films, natural fibers (e.g., wood or cotton fibers), synthetic fibers (e.g., polyester or polypropylene fibers), or a combination thereof. In some embodiments, woven and/or nonwoven fabrics are used for the liner 5. For example, the bodyside liner 5 may be formed from a meltblown or spunbonded web of polyolefin fibers. The liner 5 may also be a bonded-carded web of natural and/or synthetic fibers. The liner 5 may further be composed of a substantially hydrophobic material that is optionally treated with a surfactant or otherwise processed to impart a desired level of wettability and hydrophilicity. The surfactant may be applied by any conventional method, such as spraying, printing, brush coating, foaming, and so forth. When utilized, the surfactant may be applied to the entire liner 5 or may be selectively applied to particular sections of the liner 5, such as to the medial section along the longitudinal centerline of the diaper. The liner 5 may further include a composition that is configured to transfer to the wearer's skin for improving skin health. Suitable compositions for use on the liner 5 are described in U.S. Pat. No. 6,149,934 to Krzysik et al., which is incorporated herein in its entirety by reference thereto for all purposes.

The absorbent core 3 may be formed from a variety of materials, but typically includes a matrix of hydrophilic fibers. In one embodiment, an absorbent web is employed that contains a matrix of cellulosic fluff fibers. One type of fluff that may be used in the present invention is identified with the trade designation CR1654, available from U.S. Alliance of Childersburg, Ala., and is a bleached, highly absorbent sulfate wood pulp containing primarily softwood fibers. Airlaid webs may also be used. In an airlaying process, bundles of small fibers having typical lengths ranging from about 3 to about 19 millimeters are separated and entrained in an air supply and then deposited onto a forming screen, usually with the assistance of a vacuum supply. The randomly deposited fibers then are bonded to one another using, for example, hot air or a spray adhesive. Another type of suitable absorbent nonwoven web for the absorbent core 3 is a coform material, which may be a blend of cellulose fibers and meltblown fibers.

In some embodiments, the absorbent core 3 may contain a superabsorbent material, e.g., a water-swellable material capable of absorbing at least about 20 times its weight and, in some cases, at least about 30 times its weight in an aqueous solution containing 0.9 weight percent sodium chloride. The superabsorbent materials may be natural, synthetic and modified natural polymers and materials. In addition, the superabsorbent materials may be inorganic materials, such as silica gels, or organic compounds such as cross-linked polymers. Examples of synthetic superabsorbent material polymers include the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrrolidone), poly(vinylmorpholinone), poly(vinyl alcohol), and mixtures and copolymers thereof. Further superabsorbent materials include natural and modified natural polymers, such as hydrolyzed acrylonitrile-grafted starch, acrylic acid grafted starch, methyl cellulose, chitosan, carboxymethyl cellulose, hydroxypropyl cellulose, and the natural gums, such as alginates, xanthan gum, locust bean gum and so forth. Mixtures of natural and wholly or partially synthetic superabsorbent polymers may also be useful in the present invention. Other suitable absorbent gelling materials are disclosed in U.S. Pat. No. 3,901,236 to Assarsson et al.; U.S. Pat. No. 4,076,663 to Masuda et al.; and U.S. Pat. No. 4,286,082 to Tsubakimoto et al., which are incorporated herein in their entirety by reference thereto for all purposes.

As illustrated in FIG. 1, the diaper 1 may also include a surge layer 7 that helps to decelerate and diffuse surges or gushes of liquid that may be rapidly introduced into the absorbent core 3. Desirably, the surge layer 7 rapidly accepts and temporarily holds the liquid prior to releasing it into the storage or retention portions of the absorbent core 3. In the illustrated embodiment, for example, the surge layer 7 is interposed between an inwardly facing surface 16 of the bodyside liner 5 and the absorbent core 3. Alternatively, the surge layer 7 may be located on an outwardly facing surface 18 of the bodyside liner 5. The surge layer 7 is typically constructed from highly liquid-permeable materials. Suitable materials may include porous woven materials, porous nonwoven materials, and apertured films. Some examples include, without limitation, flexible porous sheets of polyolefin fibers, such as polypropylene, polyethylene or polyester fibers; webs of spunbonded polypropylene, polyethylene or polyester fibers; webs of rayon fibers; bonded carded webs of synthetic or natural fibers or combinations thereof. Other examples of suitable surge layers 7 are described in U.S. Pat. No. 5,486,166 to Ellis, et al. and U.S. Pat. No. 5,490,846 to Ellis et al., which are incorporated herein in their entirety by reference thereto for all purposes.

Besides the above-mentioned components, the diaper 1 may also contain various other components as is known in the art. For example, the diaper 1 may also contain a substantially hydrophilic tissue wrapsheet (not illustrated) that helps maintain the integrity of the airlaid fibrous structure of the absorbent core 3. The tissue wrapsheet is typically placed about the absorbent core 3 over at least the two major facing surfaces thereof, and composed of an absorbent cellulosic material, such as creped wadding or a high wet-strength tissue. The tissue wrapsheet may be configured to provide a wicking layer that helps to rapidly distribute liquid over the mass of absorbent fibers of the absorbent core 3. The wrapsheet material on one side of the absorbent fibrous mass may be bonded to the wrapsheet located on the opposite side of the fibrous mass to effectively entrap the absorbent core 3.

Furthermore, the diaper 1 may also include a ventilation layer (not shown) that is positioned between the absorbent core 3 and the outer cover 17. When utilized, the ventilation layer may help insulate the outer cover 17 from the absorbent core 3, thereby reducing dampness in the outer cover 17. Examples of such ventilation layers may include breathable laminates (e.g., nonwoven web laminated to a breathable film), such as described in U.S. Pat. No. 6,663,611 to Blaney, et al., which is incorporated herein in its entirety by reference thereto for all purpose.

In some embodiments, the diaper 1 may also include a pair of ears (not shown) that extend from the side edges 22 of the diaper 1 into one of the waist regions. The ears may be integrally formed with a selected diaper component. For example, the ears may be integrally formed with the outer cover 17 or from the material employed to provide the top surface. In alternative configurations, the ears may be provided by members connected and assembled to the outer cover 17, the top surface, between the outer cover 17 and top surface, or in various other configurations.

As representatively illustrated in FIG. 1, the diaper 1 may also include a pair of containment flaps 12 that are configured to provide a barrier and to contain the lateral flow of body exudates. The containment flaps 12 may be located along the laterally opposed side edges 22 of the bodyside liner 5 adjacent the side edges of the absorbent core 3. The containment flaps 12 may extend longitudinally along the entire length of the absorbent core 3, or may only extend partially along the length of the absorbent core 3. When the containment flaps 12 are shorter in length than the absorbent core 3, they may be selectively positioned anywhere along the side edges 22 of diaper 1 in a crotch region 10. In one embodiment, the containment flaps 12 extend along the entire length of the absorbent core 3 to better contain the body exudates. Such containment flaps 12 are generally well known to those skilled in the art. For example, suitable constructions and arrangements for the containment flaps 12 are described in U.S. Pat. No. 4,704,116 to Enloe, which is incorporated herein in its entirety by reference thereto for all purposes.

The diaper 1 may include various elastic or stretchable materials, such as a pair of leg elastic members 6 affixed to the side edges 22 to further prevent leakage of body exudates and to support the absorbent core 3. In addition, a pair of waist elastic members 8 may be affixed to longitudinally opposed waist edges 15 of the diaper 1. The leg elastic members 6 and the waist elastic members 8 are generally adapted to closely fit about the legs and waist of the wearer in use to maintain a positive, contacting relationship with the wearer and to effectively reduce or eliminate the leakage of body exudates from the diaper 1. As used herein, the terms “elastic” and “stretchable” include any material that may be stretched and return to its original shape when relaxed. Suitable polymers for forming such materials include, but are not limited to, block copolymers of polystyrene, polyisoprene and polybutadiene; copolymers of ethylene, natural rubbers and urethanes; etc. Particularly suitable are styrene-butadiene block copolymers sold by Kraton Polymers of Houston, Tex. under the trade name Kraton®. Other suitable polymers include copolymers of ethylene, including without limitation ethylene vinyl acetate, ethylene methyl acrylate, ethylene ethyl acrylate, ethylene acrylic acid, stretchable ethylene-propylene copolymers, and combinations thereof. Also suitable are coextruded composites of the foregoing, and elastomeric staple integrated composites where staple fibers of polypropylene, polyester, cotton and other materials are integrated into an elastomeric meltblown web. Certain elastomeric single-site or metallocene-catalyzed olefin polymers and copolymers are also suitable for the side panels.

The diaper 1 may also include one or more fasteners 20. For example, two flexible fasteners 20 are illustrated in FIG. 1 on opposite side edges of waist regions to create a waist opening and a pair of leg openings about the wearer. The shape of the fasteners 20 may generally vary, but may include, for instance, generally rectangular shapes, square shapes, circular shapes, triangular shapes, oval shapes, linear shapes, and so forth. The fasteners may include, for instance, a hook material. In one particular embodiment, each fastener 20 includes a separate piece of hook material affixed to the inside surface of a flexible backing.

The various regions and/or components of the diaper 1 may be assembled together using any known attachment mechanism, such as adhesive, ultrasonic, thermal bonds, etc. Suitable adhesives may include, for instance, hot melt adhesives, pressure-sensitive adhesives, and so forth. When utilized, the adhesive may be applied as a uniform layer, a patterned layer, a sprayed pattern, or any of separate lines, swirls or dots. In some embodiments, the exothermic coating of the present invention may serve the dual purposes of generating heat and also acting as the adhesive. For example, the binder of the exothermic coating may bond together one or more regions of the diaper 1. In the illustrated embodiment, for example, the outer cover 17 and bodyside liner 5 are assembled to each other and to the absorbent core 3 using an adhesive. Alternatively, the absorbent core 3 may be connected to the outer cover 17 using conventional fasteners, such as buttons, hook and loop type fasteners, adhesive tape fasteners, and so forth. Similarly, other diaper components, such as the leg elastic members 6, waist elastic members 8 and fasteners 20, may also be assembled into the diaper 1 using any attachment mechanism.

Although various configurations of a diaper have been described above, it should be understood that other diaper configurations are also included within the scope of the present invention. For instance, other suitable diaper configurations are described in U.S. Pat. No. 4,798,603 to Mever et al.; U.S. Pat. No. 5,176,668 to Bemardin; U.S. Pat. No. 5,176,672 to Bruemmer et al.; U.S. Pat. No. 5,192,606 to Proxmire et al.; and U.S. Pat. No. 5,509,915 to Hanson et al., as well as U.S. Patent application Pub. No. 2003/120253 to Wentzel, et al., all of which are incorporated herein in their entirety by reference thereto for all purposes. In addition, the present invention is by no means limited to diapers. In fact, any other absorbent article may be formed in accordance with the present invention, including, but not limited to, other personal care absorbent articles, such as training pants, absorbent underpants, adult incontinence products, feminine hygiene products (e.g., sanitary napkins), swim wear, baby wipes, and so forth; medical absorbent articles, such as garments, fenestration materials, underpads, bandages, absorbent drapes, and medical wipes; food service wipers; clothing articles; and so forth. Several examples of such absorbent articles are described in U.S. Pat. No. 5,197,959 to Buell; U.S. Pat. No. 5,085,654 to Buell; U.S. Pat. No. 5,634,916 to Lavon, et al.; U.S. Pat. No. 5,569,234 to Buell, et al.; U.S. Pat. No. 5,716,349 to Taylor, et al.; U.S. Pat. No. 4,950,264 to Osborn, III; U.S. Pat. No. 5,009,653 to Osborn, III; U.S. Pat. No. 5,509,914 to Osborn, III; U.S. Pat. No. 5,649,916 to DiPalma, et al.; U.S. Pat. No. 5,267,992 to Van Tillburg; U.S. Pat. No. 4,687,478 to Van Tillburg; U.S. Pat. No. 4,285,343 to McNair; U.S. Pat. No. 4,608,047 to Mattingly; U.S. Pat. No. 5,342,342 to Kitaoka; U.S. Pat. No. 5,190,563 to Herron, et al.; U.S. Pat. No. 5,702,378 to Widlund, et al.; U.S. Pat. No. 5,308,346 to Sneller, et al.; U.S. Pat. No. 6,110,158 to Kielpikowski; U.S. Pat. No. 6,663,611 to Blaney, et al.; and WO 99/00093 to Patterson, et al., which are incorporated herein in their entirety by reference thereto for all purposes.

Regardless of the manner in which the absorbent article is formed, a warmth-providing substrate may be employed in accordance with the present invention. Besides the materials referenced above, any other material may generally be used to form the warmth-providing substrate. For instance, nonwoven fabrics, woven fabrics, knit fabrics, paper web, film, foams, etc., may be applied with the exothermic coating. When utilized, the nonwoven fabrics may include, but are not limited to, spunbonded webs (apertured or non-apertured), meltblown webs, bonded carded webs, air-laid webs, coform webs, hydraulically entangled webs, and so forth. Typically, the polymers used to form the substrate have a softening or melting temperature that is higher than the temperature needed to evaporate water. One or more components of such polymers may have, for instance, a softening temperature of from about 100° C. to about 400° C., in some embodiments from about 110° C. to about 300° C., and in some embodiments, from about 120° C. to about 250° C. Examples of such polymers may include, but are not limited to, synthetic polymers (e.g., polyethylene, polypropylene, polyethylene terephthalate, nylon 6, nylon 66, KEVLAR™, syndiotactic polystyrene, liquid crystalline polyesters, etc.); cellulosic polymers (softwood pulp, hardwood pulp, thermomechanical pulp, etc.); combinations thereof; and so forth.

Referring again to FIG. 1, the warmth-providing substrate may be incorporated to any component of the diaper 1, including the outer cover 17, the bodyside liner 5, the absorbent core 3, the tissue wrapsheet (not shown), the surge layer 7, the ventilation layer (not shown), and/or any other portion of the diaper 1. In one particular embodiment, for example, the warmth-providing substrate is used to form all or a portion of the outer cover 17 and/or ventilation layer. In this manner, the substrate may be located adjacent to or near a wearer's skin to mitigate the damp or cooling effect often caused by the condensation of water vapor on the surface of the outer cover 17.

Besides providing warmth, the substrate may also fulfill other functions of the layer into which it is incorporated. For example, when used as the outer cover 17 or some other component of the diaper 1, the warmth-providing substrate may be “breathable” to permit the flow of vapors from the absorbent core 3 and also to prevent liquid exudates from escaping therefrom. This permits the flow of water vapor and air for activating the exothermic reaction, but prevents an excessive amount of liquids from contacting the warmth-providing substrate, which could either suppress the reaction or result in an excessive amount of heat that overly warms or burns the user.

To form the warmth-providing substrate for use in the absorbent article, some or all of the substrate is generally coated with an exothermic coating. The exothermic coating contains a metal that oxidizes in the presence of oxygen and moisture. Examples of such metals include, but are not limited to, iron, zinc, aluminum, magnesium, and so forth. Although not required, the metal may be initially provided in powder form to facilitate handling and to reduce costs. Various methods for removing impurities from a crude metal (e.g. iron) to form a powder include, for example, wet processing techniques, such as solvent extraction, ion exchange, and electrolytic refining for separation of metallic elements; hydrogen gas (H₂) processing for removal of gaseous elements, such as oxygen and nitrogen; floating zone melting refining method. Using such techniques, the metal purity may be at least about 95%, in some embodiments at least about 97%, and in some embodiments, at least about 99%. The particle size of the metal powder may also be less than about 500 micrometers, in some embodiments less than about 100 micrometers, and in some embodiments, less than about 50 micrometers. The use of such small particles may enhance the contact surface of the metal with air, thereby improving the likelihood and efficiency of the desired exothermal reaction. The concentration of the metal powder employed may generally vary depending on the nature of the metal powder, and the desired extent of the exothermal/oxidation reaction. In most embodiments, the metal powder is present in the exothermic coating in an amount from about 40 wt. % to about 95 wt. %, in some embodiments from about 50 wt. % to about 90 wt. %, and in some embodiments, from about 60 wt. % to about 80 wt. %.

In addition to an oxidizable metal, a carbon component may also be utilized in the exothermic coating of the present invention. Without intending to be limited in theory, it is believed that such a carbon component promotes the oxidation reaction of the metal and acts as a catalyst for generating heat. The carbon component may be activated carbon, carbon black, graphite, and so forth. When utilized, activated carbon may be formed from sawdust, wood, charcoal, peat, lignite, bituminous coal, coconut shells, etc. Some suitable forms of activated carbon and techniques for formation thereof are described in U.S. Pat. No. 5,693,385 to Parks; U.S. Pat. No. 5,834,114 to Economy, et al.; U.S. Pat. No. 6,517,906 to Economy, et al.; U.S. Pat. No. 6,573,212 to McCrae, et al., as well as U.S. Patent application Publication Nos. 2002/0141961 to Falat. et al. and 2004/0166248 to Hu, et al., all of which are incorporated herein in their entirety by reference thereto for all purposes.

The exothermic coating may also employ a binder for enhancing the durability of the exothermic coating when applied to a substrate. The binder may also serve as an adhesive for bonding one substrate to another substrate. For example, the binder may be used as an adhesive for laminating a nonwoven material to a breathable film, such as used in forming the outer cover of a diaper. Generally speaking, any of a variety of binders may be used in the exothermic coating of the present invention. Suitable binders may include, for instance, those that become insoluble in water upon crosslinking. Crosslinking may be achieved in a variety of ways, including by reaction of the binder with a polyfunctional crosslinking agent. Examples of such crosslinking agents include, but are not limited to, dimethylol urea melamine-formaldehyde, urea-formaldehyde, polyamide epichlorohydrin, etc.

In some embodiments, a polymer latex may be employed as the binder. The polymer suitable for use in the lattices typically has a glass transition temperature of about 30° C. or less so that the flexibility of the resulting substrate is not substantially restricted. Moreover, the polymer also typically has a glass transition temperature of about −25° C. or more to minimize the tackiness of the polymer latex. For instance, in some embodiments, the polymer has a glass transition temperature from about −15° C. to about 15° C., and in some embodiments, from about −10° C. to about 0° C. For instance, some suitable polymer lattices that may be utilized in the present invention may be based on polymers such as, but are not limited to, styrene-butadiene copolymers, polyvinyl acetate homopolymers, vinyl-acetate ethylene copolymers, vinyl-acetate acrylic copolymers, ethylene-vinyl chloride copolymers, ethylene-vinyl chloride-vinyl acetate terpolymers, acrylic polyvinyl chloride polymers, acrylic polymers, nitrile polymers, and any other suitable anionic polymer latex polymers known in the art. The charge of the polymer lattices described above may be readily varied, as is well known in the art, by utilizing a stabilizing agent having the desired charge during preparation of the polymer latex. Specific techniques for a carbon/polymer latex system are described in more detail in U.S. Pat. No. 6,573,212 to McCrae, et al. Commercially available activated carbon/polymer latex systems that may be used in the present invention include Nuchar® PMA, DPX-8433-68A, and DPX-8433-68B, all of which are available from MeadWestvaco Corp of Stamford, Conn.

Although polymer lattices may be effectively used as binders in the present invention, such compounds sometimes result in a reduction in drapability and an increase in residual odor. Thus, the present inventor has discovered that water-soluble organic polymers may also be employed as binders to alleviate such concerns. For example, one class of water-soluble organic polymers found to be suitable in the present invention is polysaccharides and derivatives thereof. Polysaccharides are polymers containing repeated carbohydrate units, which may be cationic, anionic, nonionic, and/or amphoteric. In one particular embodiment, the polysaccharide is a nonionic, cationic, anionic, and/or amphoteric cellulosic ether. Suitable nonionic cellulosic ethers may include, but are not limited to, alkyl cellulose ethers, such as methyl cellulose and ethyl cellulose; hydroxyalkyl cellulose ethers, such as hydroxyethyl cellulose, hydroxypropyl cellulose, hydroxypropyl hydroxybutyl cellulose, hydroxyethyl hydroxypropyl cellulose, hydroxyethyl hydroxybutyl cellulose and hydroxyethyl hydroxypropyl hydroxybutyl cellulose; alkyl hydroxyalkyl cellulose ethers, such as methyl hydroxyethyl cellulose, methyl hydroxypropyl cellulose, ethyl hydroxyethyl cellulose, ethyl hydroxypropyl cellulose, methyl ethyl hydroxyethyl cellulose and methyl ethyl hydroxypropyl cellulose; and so forth.

Suitable cellulosic ethers may include, for instance, those available from Akzo Nobel of Stamford, Conn. under the name “BERMOCOLL.” Still other suitable cellulosic ethers are those available from Shin-Etsu Chemical Co., Ltd. of Tokyo, Japan under the name “METOLOSE”, including METOLOSE Type SM (methycellulose), METOLOSE Type SH (hydroxypropylmethyl cellulose), and METOLOSE Type SE (hydroxyethylmethyl cellulose). One particular example of a suitable nonionic cellulosic ether is ethyl hydroxyethyl cellulose having a degree of ethyl substitution (DS) of 0.8 to 1.3 and a molar substitution (MS) of hydroxyethyl of 1.9 to 2.9. The degree of ethyl substitution represents the average number of hydroxyl groups present on each anhydroglucose unit that have been reacted, which may vary between 0 and 3. The molar substitution represents the average number of hydroxethyl groups that have reacted with each anhydroglucose unit. One such cellulosic ether is BERMOCOLL E 230FQ, which is an ethyl hydroxyethyl cellulose commercially available from Akzo Nobel. Other suitable cellulosic ethers are also available from Hercules, Inc. of Wilmington, Del. under the name “CULMINAL.”

The concentration of the carbon component and/or binder in the exothermic coating may generally vary based on the desired properties of the substrate. For example, the amount of the carbon component is generally tailored to facilitate the oxidation/exothermic reaction without adversely affecting other properties of the substrate. Typically, the carbon component is present in the exothermic coating in an amount about 0.01 wt. % to about 20 wt. %, in some embodiments from about 0.1 wt. % to about 15 wt. %, and in some embodiments, from about 1 wt. % to about 12 wt. %. In addition, although relatively high binder concentrations may provide better physical properties for the exothermic coating, they may likewise have an adverse effect on other properties, such as the absorptive capacity of the substrate to which it is applied. Conversely, relatively low binder concentrations may reduce the ability of the exothermic coating to remain affixed on the substrate. Thus, in most embodiments, the binder is present in the exothermic coating in an amount from about 0.01 wt. % to about 20 wt. %, in some embodiments from about 0.1 wt. % to about 10 wt. %, and in some embodiments, from about 0.5 wt. % to about 5 wt. %.

Still other components may also be employed in the exothermic coating of the present invention. For example, as is well known in the art, an electrolytic salt may be employed to react with and remove any passivating oxide layer(s) that might otherwise prevent the metal from oxidizing. Suitable electrolytic salts may include, but are not limited to, alkali halides or sulfates, such as sodium chloride, potassium chloride, etc.; alkaline halides or sulfates, such as calcium chloride, magnesium chloride, etc., and so forth. When employed, the electrolytic salt is typically present in the exothermic coating in an amount from about 0.01 wt. % to about 10 wt. %, in some embodiments from about 0.1 wt. % to about 8 wt. %, and in some embodiments, from about 1 wt. % to about 6 wt. %.

In addition, particles may also be employed in the exothermic coating that act as moisture retainers. That is, prior to the oxidation/exothermic reaction, these particles may retain moisture. However, after the reaction has proceeded to a certain extent and the moisture concentration is reduced, the particles may release the moisture to allow the reaction to continue. Besides acting as a moisture retainer, the particles may also provide other benefits to the exothermic coating of the present invention. For example, the particles may alter the black color normally associated with the carbon component and/or metal powder. When utilized, the size of the moisture-retaining particles may be less than about 500 micrometers, in some embodiments less than about 100 micrometers, and in some embodiments, less than about 50 micrometers. Likewise, the particles may be porous. Without intending to be limited by theory, it is believed that porous particles may provide a passage for air and/or water vapors to better contact the metal powder. For example, the particles may have pores/channels with a mean diameter of greater than about 5 angstroms, in some embodiments greater than about 20 angstroms, and in some embodiments, greater than about 50 angstroms. The surface area of such particles may also be greater than about 15 square meters per gram, in some embodiments greater than about 25 square meters per gram, and in some embodiments, greater than about 50 square meters per gram. Surface area may be determined by the physical gas adsorption (B.E.T.) method of Bruanauer, Emmet, and Teller, Journal of American Chemical Society, Vol. 60, 1938, p. 309, with nitrogen as the adsorption gas.

In one particular embodiment, porous carbonate particles (e.g., calcium carbonate) are used to retain moisture and also to alter the black color normally associated with activated carbon and/or metal powder. Such a color change may be more aesthetically pleasing to a user, particularly when the coating is employed on substrates designed for consumer/personal use. Suitable white calcium carbonate particles are commercially available from Omya, Inc. of Proctor, Vt. Still other suitable particles that may retain moisture include, but are not limited to, silicates, such as calcium silicate, alumina silicates (e.g., mica powder, clay, etc.), magnesium silicates (e.g., talc), quartzite, calcium silicate fluorite, etc.; alumina; silica; and so forth. The concentration of the particles may generally vary depending on the nature of the particles, and the desired extent of exothermic reaction and color alteration. For instance, the particles may be present in the exothermic coating in an amount from about 0.01 wt. % to about 30 wt. %, in some embodiments from about 0.1 wt. % to about 20 wt. %, and in some embodiments, from about 1 wt. % to about 15 wt. %.

In addition to the above-mentioned components, other components, such as surfactants, pH adjusters, dyes/pigments, etc., may also be included in the exothermic coating of the present invention. Although not required, such additional components typically constitute less than about 5 wt. %, in some embodiments less than about 2 wt. %, and in some embodiments, from about 0.001 wt. % to about 1 wt. % of the exothermic coating.

To apply the exothermic coating of the present invention to a substrate, the components may initially be dissolved or dispersed in a solvent. For example, one or more of the above-mentioned components may be mixed with a solvent, either sequentially or simultaneously, to form a coating formulation that may be easily applied to a substrate. Any solvent capable of dispersing or dissolving the components is suitable, for example water; alcohols such as ethanol or methanol; dimethylformamide; dimethyl sulfoxide; hydrocarbons such as pentane, butane, heptane, hexane, toluene and xylene; ethers such as diethyl ether and tetrahydrofuran; ketones and aldehydes such as acetone and methyl ethyl ketone; acids such as acetic acid and formic acid; and halogenated solvents such as dichloromethane and carbon tetrachloride; as well as mixtures thereof. In one particular embodiment, for example, water is used as the solvent so that an aqueous coating formulation is formed. The concentration of the solvent is generally high enough to inhibit oxidization of the metal prior to use. Specifically, when present in a high enough concentration, the solvent may act as a barrier to prevent air from prematurely contacting the oxidizable metal. If the amount of solvent is too small, however, the exothermic reaction may occur prematurely. Likewise, if the amount of solvent is too large, the amount of metal deposited on the substrate might be too low to provide the desired exothermal effect. Although the actual concentration of solvent (e.g., water) employed will generally depend on the type of oxidizable metal and the substrate on which it is applied, it is nonetheless typically present in an amount from about 10 wt. % to about 80 wt. %, in some embodiments from about 20 wt. % to about 70 wt. %, and in some embodiments, from about 25 wt. % to about 60 wt. % of the coating formulation.

The amount of the other components added to the coating formulation may vary depending on the amount of heat desired, the wet pick-up of the application method utilized, etc. For example, the amount of the oxidizable metal (in powder form) within the coating formulation generally ranges from about 20 wt. % to about 80 wt. %, in some embodiments from about 30 wt. % to about 70 wt. %, and in some embodiments, from about 35 wt. % to about 60 wt. %. In addition, the carbon component may constitute from about 0.1 wt. % to about 20 wt. %, in some embodiments from about 0.1 wt. % to about 15 wt. %, and in some embodiments, from about 0.2 wt. % to about 10 wt. %. of the coating formulation. Binders may constitute from about 0.01 wt. % to about 20 wt. %, in some embodiments from about 0.1 wt. % to about 15 wt. %, and in some embodiments, from about 1 wt. % to about 10 wt. % of the coating formulation. Electrolytic salts may constitute from about 0.01 wt. % to about 10 wt. %, in some embodiments from about 0.1 wt. % to about 8 wt. %, and in some embodiments, from about 1 wt. % to about 5 wt. %. of the coating formulation. Further, moisture-retaining particles may constitute from about 2 wt. % to about 30 wt. %, in some embodiments from about 3 wt. % to about 25 wt. %, and in some embodiments, from about 4 wt. % to about 10 wt. %. of the coating formulation. Other components, such as surfactants, pH adjusters, etc., may also constitute from about 0.001 wt. % to about 0.5 wt. %, in some embodiments from about 0.01 wt. % to about 0.1 wt. %, and in some embodiments from about 0.02 wt. % to about 0.08 wt. % of the coating formulation.

The solids content and/or viscosity of the coating formulation may be varied to achieve the desired amount of heat generation. For example, the coating formulation may have a solids content of from about 30% to about 80%, in some embodiments from about 40% to about 70%, and in some embodiments, from about 50% to about 60%. By varying the solids content of the coating formulation, the presence of the metal powder and other components in the exothermic coating may be controlled. For example, to form an exothermic coating with a higher level of metal powder, the coating formulation may be provided with a relatively high solids content so that a greater percentage of the metal powder is incorporated into the exothermic coating during the application process. In addition, the viscosity of the coating formulation may also vary depending on the coating method and/or type of binder employed. For instance, lower viscosities may be employed for saturation coating techniques (e.g., dip-coating), while higher viscosities may be employed for drop-coating techniques. Generally, the viscosity is less than about 2×10⁶ centipoise, in some embodiments less than about 2×10⁵ centipoise, in some embodiments less than about 2×10⁴ centipoise, and in some embodiments, less than about 2×10³ centipoise, such as measured with a Brookfield DV-1 viscometer with an LV-IV spindle. If desired, thickeners or other viscosity modifiers may be employed in the coating formulation to increase or decrease viscosity.

The coating formulation may be applied to a substrate using any conventional technique, such as bar, roll, knife, curtain, print (e.g., rotogravure), spray, slot-die, drop-coating, or dip-coating techniques. The materials that form the substrate (e.g., fibers) may be coated before and/or after incorporation into the substrate. The coating may be applied to one or both surfaces of the substrate. For example, the exothermic coating may be present on a surface of the substrate that is opposite to that facing the wearer or user to avoid the possibility of burning. In addition, the coating formulation may cover an entire surface of the substrate, or may only cover a portion of the surface. When applying the exothermic coating to multiple surfaces, each surface may be coated sequentially or simultaneously.

Regardless of the manner in which the coating is applied, the resulting coated substrate is heated to a certain temperature to remove the solvent and any moisture from the coating. For example, the coated substrate may be heated to a temperature of at least about 100° C., in some embodiments at least about 110° C., and in some embodiments, at least about 120° C. In this manner, the resulting dried exothermic coating is anhydrous, i.e., generally free of water. By minimizing the amount of moisture, the exothermic coating is less likely to react prematurely and generate heat. That is, the oxidizable metal does not generally react with oxygen unless some minimum amount of water is present. Thus, the exothermic coating may remain inactive until placed in the vicinity of moisture (e.g., next to an absorbent layer) during use. It should be understood, however, that relatively small amounts of water may still be present in the exothermic coating without causing a substantial exothermic reaction. In some embodiments, for example, the exothermic coating contains water in an amount less than about 0.5% by weight, in some embodiments less than about 0.1% by weight, and in some embodiments, less than about 0.01% by weight.

The solids add-on level of the exothermic coating may also be varied as desired. The “solids add-on level” is determined by subtracting the weight of the untreated substrate from the weight of the treated substrate (after drying), dividing this calculated weight by the weight of the untreated substrate, and then multiplying by 100%. Lower add-on levels may optimize certain properties (e.g., absorbency), while higher add-on levels may optimize heat generation. In some embodiments, for example, the add-on level is from about 20% to about 600%, in some embodiments from about 50% to about 500%, and in some embodiments, from about 100% to about 400%. The thickness of the exothermic coating may also vary. For example, the thickness may range from about 0.001 millimeters to about 0.4 millimeters, in some embodiments, from about 0.01 millimeters to about 0.30 millimeters, and in some embodiments, from about 0.01 millimeters to about 0.20 millimeters. Such a relatively thin coating may enhance the flexibility of the substrate, while still providing uniform heating.

To maintain absorbency, porosity, flexibility, and/or some other characteristic of the substrate, it may sometimes be desired to apply the exothermic coating so as to cover less than 100%, in some embodiments from about 10% to about 80%, and in some embodiments, from about 20% to about 60% of the area of one or more surfaces of the substrate. For instance, in one particular embodiment, the exothermic coating is applied to the substrate in a preselected pattern (e.g., reticular pattern, diamond-shaped grid, dots, and so forth). Although not required, such a patterned exothermic coating may provide sufficient warming to the substrate without covering a substantial portion of the surface area of the substrate. This may be desired to optimize flexibility, absorbency, or other characteristics of the substrate. It should be understood, however, that the coating may also be applied uniformly to one or more surfaces of the substrate. In addition, a patterned exothermic coating may also provide different functionality to each zone. For example, in one embodiment, the substrate is treated with two or more patterns of coated regions that may or may not overlap. The regions may be on the same or different surfaces of the substrate. In one embodiment, one region of a substrate is coated with a first exothermic coating, while another region is coated with a second exothermic coating. If desired, one region may provide a different amount of heat than another region.

Besides having functional benefits, the coated substrate may also have various aesthetic benefits as well. For example, although containing activated carbon, the coated substrate may be made without the black color commonly associated with activated carbon. In one embodiment, white or light-colored particles (e.g., calcium carbonate, titanium dioxide, etc.) are employed in the exothermic coating so that the resulting substrate has a grayish or bluish color. In addition, various pigments and/or dyes may be employed to alter the color of the exothermic coating. The substrate may also be applied with patterned regions of the exothermic coating to form a substrate having differently colored regions.

Prior to use, the exothermic coating is substantially free from water, and thus, heat is not generated until moisture is provided. Because the coated substrate is generally free of water, it need not be specially packaged or sealed to prevent contact with air. Further, the small amount of moisture generally present in air is typically insufficient to cause the exothermic reaction to proceed to any significant extent. Nevertheless, it may be desired in some cases to package the substrate within a substantially liquid-impermeable material (vapor-permeable or vapor-impermeable) prior to use to ensure that it does not inadvertently contact enough moisture to initiate the exothermic reaction. To activate the exothermic coating, moisture is applied during the normal course of use (e.g., absorbent articles) or as an additional activation step. When applying moisture in an additional activation step, various techniques may be employed, including spraying, dipping, coating, dropping (e.g., using a syringe), etc. Likewise, moisture simply absorbed from the surrounding environment may activate the composition. Although the amount of moisture applied may vary depending on the reaction conditions and the amount of heat desired, moisture may sometimes be added in an amount from about 20 wt. % to about 500 wt. %, and in some embodiments, from about 50 wt. % to about 200 wt. %, of the weight of the amount of oxidizable metal present in the coating. In any event, a sufficient amount of moisture is present to activate an exothermic, electrochemical reaction between the electrochemically oxidizable element (e.g., metal powder) and the electrochemically reducible element (e.g., oxygen).

Other layers may also be employed to improve the exothermic properties of the coated substrate. For example, a first coated substrate may be employed in conjunction with a second coated substrate. The substrates may function together to provide heat to a surface, or may each provide heat to different surfaces. In addition, substrates may be employed that are not applied with the exothermic coating of the present invention, but instead applied with a coating that simply facilitates the reactivity of the exothermic coating. For example, a substrate may be used near or adjacent to the coated substrate of the present invention that includes a coating of moisture-retaining particles. As described above, the moisture-retaining particles may retain and release moisture for activating the exothermic reaction.

The exothermic coating of the present invention may cause one or more regions of the absorbent article to achieve a temperature that is elevated above the ambient temperature. In many cases, this elevated temperature may prohibit any water vapor passing through the article (e.g., via a breathable layer) from condensing on the surface, thereby reducing the cold, damp feel often experienced by users of breathable absorbent articles. Typically, the exothermic coating may cause one or more regions of the absorbent article to achieve a temperature that is at least about 1° C., in some embodiments at least about 2° C., and in some embodiments, at least about 3° C. above the ambient temperature. Such an elevated temperature may sometimes range from about 30° C. to about 60° C., in some embodiments from about 35° C. to about 50° C., and in some embodiments from about 37° C. to about 43° C. Desirably, the elevated temperature is also maintained for at least about 1 hour, in some embodiments at least about 2 hours, in some embodiments at least about 4 hours, and in some embodiments, at least about 10 hours (e.g., for overnight use).

The present invention may be better understood with reference to the following example.

### EXAMPLE

The ability to form a warmth-providing substrate for use in an absorbent article in accordance with the present invention was demonstrated. The exothermic coating was prepared as follows. In a 400-milliliter pyrex beaker, 5.0 grams of Bermocoll E230 FQ (ethyl hydroxyethyl cellulose, available from Akzo Nobel) and 12.5 grams of sodium chloride (Mallinckrodt) were added to 150.4 grams of warm (ca. 58° C.) distilled water while stirring. The formulation was then cooled to ca. 18° C. with an ice bath. The resulting formulation had a solids content of 10.4% and a viscosity of 735 centipoise (measured by Brookfield DV-1 viscometer with LV-2 spindle at 12 RPM). Thereafter, 104.6 grams of an aqueous slurry of calcium carbonate particles were added to the formulation while stirring. The aqueous calcium carbonate slurry was obtained from Omya, Inc. under the name “XC4900” and had a solids content of 28.3%. After adding the calcium carbonate slurry, the formulation had a solids content of 17.4% and a viscosity of 1042 centipoise. Thereafter, 212.8 grams of iron powder and 25.2 grams of activated carbon powder were then added to the formulation. The iron powder was obtained from North American Höganäs under the name “AC-325” (-325 mesh iron powder), and the activated carbon was obtained from MeadWestvaco Corp. under the name “Nuchar SA-20.” The final solids content of the formulation was 58.3% and the final viscosity was 185,200 centipoise. The calculated concentration of each component of the aqueous formulation is set forth below in Table 1.

TABLE 1
Components of the Aqueous Formulation

| Component | Calculated Amount |
|---|---|
| Iron | 41.7% |
| Activated Carbon | 4.9% |
| Calcium Carbonate | 5.8% |
| Bermocoll E230 FQ | 1.0% |
| Sodium Chloride | 2.5% |
| Water | 44.1% |

The aqueous formulation was then uniformly coated onto one side of a fabric sample using a #60 single wound metering rod. The fab0ric sample was a flannel-like fabric available from Kimberly-Clark under the name DUStop™. The fabric had a size of 8 inches by 11.5 inches, and was a thermally bonded laminate containing a meltblown interior layer (0.5 ounces per square yard (osy) basis weight) and three spundbond layers (1.5 osy basis weight) formed from polyethylene/polypropylene side-by-side bicomponent fibers. After applying the aqueous formulation, the coated fabric was then dried in a forced air oven at 110° C. for about 10 minutes. The concentration of the components of the exothermic coating was then calculated from the initial fabric weight (10.5 grams), the dry coated fabric weight (24.2 grams), and the composition of the aqueous formulation. The results are set forth below in Table 2.

TABLE 2
Components of the Exothermic Coating

| Component | Calculated Amount |
|---|---|
| Iron | 74.6% |
| Activated Carbon | 8.8% |
| Calcium Carbonate | 10.4% |
| Bermocoll E230 FQ | 1.8% |
| Sodium Chloride | 4.4% |
| Solids Add-On Level | 130.5% |

To test the effectiveness of the coated fabric in providing warmth, a three-inch diameter circular piece of the coated fabric (2.41 grams) was placed over a cast aluminum flange type cup (50.8 mm deep) that was partially filled with 100 milliliters of distilled water. A mechanical seal and neoprene gasket were used to seal the fabric piece to the cup above the water level, with the uncoated side of the fabric facing the water and the coated side of the fabric facing the air. A thermocouple wired to a data collection device was attached to the coated side of the fabric to monitor the temperature at 3-second intervals. A small piece of Scotch® tape and the weight of a penny were used to keep the thermocouple in place during the 6-hour experiment.

Besides the above-described sample (identified hereinafter as “Sample 1”), various other samples were also tested. Specifically, another Dustop™ sample fabric was applied with an exothermic coating in the manner set forth above, but was also positioned adjacent to two separate spunbond-film laminates (identified hereinafter as “Sample 2”). The first laminate was placed on top of the iron-coated fabric, with the film side of one laminate contacting the iron-coated side of the fabric. The second laminate was placed on top of the first laminate, with the film side of the second laminate contacting the spunbond side of the first laminate. The spunbond web of each laminate had a basis weight of 0.5 ounces per square yard, was formed from polypropylene, and was necked 50% prior to lamination. The breathable film of each laminate was a microporous filmed formed from 33 wt. % of an S-EP-S elastomeric block copolymer available from Kuraray Company, Ltd. of Okayama, Japan under the trade name SEPTON®; 16.75 wt. % of linear low density polyethylene; and 50.25 wt. % of a calcium carbonate filler. The film was adhesively laminated to the spunbond web. Methods for forming such a spunbond/film laminate are described in U.S. Pat. No. 6,794,024 to Walton, et al.

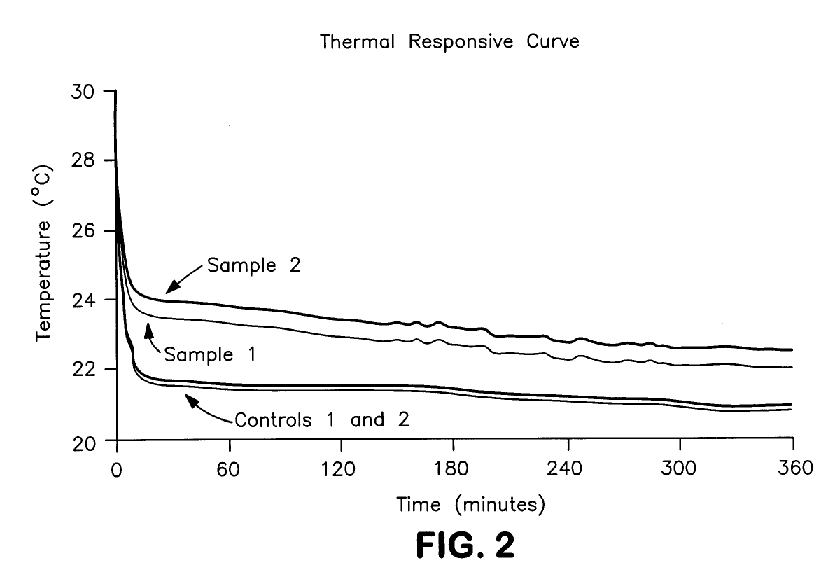

Further, first and second control samples (Control 1 and Control 2) were also tested that were identical to Samples 1 and 2, respectively, except that the0 control samples did not contain the exothermic coating. The thermal curves for the tested sample are provided in FIG. 2. In addition, the breathability of the Control 1, Sample 1, and Sample 2 (including the spunbond/film laminate) was also determined. The breathability of Control 1, Sample 1, and Sample 2 was thus determined to be approximately 16,923; 12,887; and 514 g/m²/24 hours; respectively.

While the invention has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is :

1. An absorbent article that comprises a substantially liquid-impermeable layer, a liquid-permeable layer, and an absorbent positioned between said substantially liquid-impermeable layer and said liquid-permeable layer, the absorbent article further comprising an exothermic coating that is formed from an oxidizable metal powder and is capable of activation in the presence of oxygen and moisture to generate heat, wherein said exothermic coating is generally free of water prior to activation.

2. The absorbent article of claim 1, wherein said substantially liquid-impermeable layer, said liquid-permeable layer, said absorbent, or combinations thereof, comprise said exothermic coating.

3. The absorbent article of claim 1, wherein said substantially liquid-impermeable layer comprises said exothermic coating.

4. The absorbent article of claim 1, wherein said substantially liquid-impermeable layer is breathable.

5. The absorbent article of claim 4, wherein said substantially liquid-impermeable layer has a WVTR of at least about 100 grams per square meter per 24 hours.

6. The absorbent article of claim 4, wherein said substantially liquid-impermeable layer has a WVTR of from about 500 to about 20,000 grams per square meter per 24 hours.

7. The absorbent article of claim 4, wherein said substantially liquid-impermeable layer has a WVTR of from about 1,000 to about 15,000 grams per square meter per 24 hours.

8. The absorbent article of claim 4, wherein said substantially liquid-impermeable layer is formed from a nonwoven web laminated to a breathable film.

9. The absorbent article of claim 8, wherein said exothermic coating helps adhere said nonwoven web material to said breathable film.

10. The absorbent article of claim 1, wherein the absorbent article comprises two or more substantially liquid-impermeable layers, at least one of which comprises said exothermic coating.

11. The absorbent article of claim 1, wherein said exothermic coating is present at a solids add-on level of from about 20% to about 600%.

12. The absorbent article of claim 1, wherein said exothermic coating is present at a solids add-on level of from about 100% to about 400%.

13. The absorbent article of claim 1, wherein said exothermic coating further comprises a carbon component, binder, and electrolytic salt.

14. The absorbent article of claim 13, wherein said carbon component is activated carbon.

15. The absorbent article of claim 1, wherein said metal powder contains iron, zinc, aluminum, magnesium, or combinations thereof.

16. The absorbent article of claim 1, wherein said metal powder constitutes from about 40 wt. % to about 95 wt. % of said exothermic coating.

17. The absorbent article of claim 1, wherein upon activation of said exothermic coating, one or more surfaces of said absorbent article reaches a temperature that is elevated above ambient temperature.

18. The absorbent article of claim 17, wherein said elevated temperature is at least about 1° C. above ambient temperature.

19. The absorbent article of claim 17, wherein said elevated temperature is at least about 2° C. above ambient temperature.

20. A personal care absorbent article that comprises:
a liquid-permeable liner;
a breathable outer cover;
an absorbent positioned between said liner and said outer cover; and
optionally, a ventilation layer positioned between said breathable outer cover and said absorbent;
wherein said breathable outer cover, said ventilation layer, or both, comprise an exothermic coating that is formed from an oxidizable metal powder and is capable of activation in the presence of oxygen and moisture to generate heat, wherein said exothermic coating is generally free of water prior to activation.

21. The personal care absorbent article of claim 20, wherein said breathable outer cover is formed from a nonwoven web laminated to a breathable film.

22. The personal care absorbent article of claim 20, wherein the exothermic coating is present at a solids add-on level of from about 100% to about 400%.

23. The personal care absorbent article of claim 20, wherein said exothermic coating further comprises a carbon component, binder, and electrolytic salt.

24. The personal care absorbent article of claim 20, wherein said metal powder constitutes from about 40 wt. % to about 95 wt. % of said exothermic coating.

25. The personal care absorbent article of claim 20, wherein said breathable outer cover, said ventilation layer, or both, have a WVTR of from about 5,000 to about 14,000 grams per square meter per 24 hours.

26. A diaper that comprises:
a liquid-permeable bodyside liner;
a breathable outer cover;
an absorbent positioned between said liner and said outer cover;
a surge layer positioned between said liner and said absorbent; and
optionally, a ventilation layer positioned between said outer cover and said absorbent;
wherein said breathable outer cover, said ventilation layer, or both, comprise an exothermic coating that is formed from an oxidizable metal powder, carbon component, binder, and metal halide, and wherein said exothermic coating is capable of activation in the presence of oxygen and moisture to generate heat, wherein said exothermic coating is generally free of water prior to activation.

27. The diaper of claim 26, wherein said breathable outer cover is formed from a nonwoven web material laminated to a breathable film.

28. The diaper of claim 26, wherein said exothermic coating is present at a solids add-on level of from about 20% to about 600%.

29. The diaper of claim 26, wherein said metal powder constitutes from about 40 wt. % to about 95 wt. % of said exothermic coating.

30. The diaper of claim 26, wherein said breathable outer cover, said ventilation layer, or both, have a WVTR of from about 1,000 to about 15,000 grams per square meter per 24 hours.
